(19) United States
(12) Patent Application Publication
(10) Pub. No.: US 2015/0087539 A1
(43) Pub. Date: Mar. 26, 2015

(54) METHOD FOR PREDICTING OUTCOME OF CANCER IN A SUBJECT

(71) Applicant: Juan Carlos Lacal Sanjuan, Madrid (ES)

(72) Inventor: Juan Carlos Lacal Sanjuan, Madrid (ES)

(73) Assignee: TRASLATIONAL CANCER DRUGS PHARMA, S.L., Valladolid (ES)

(21) Appl. No.: 14/232,860

(22) PCT Filed: Jul. 13, 2012
(86) PCT No.: PCT/EP12/63751

§ 371 Date: Sep. 2, 2014

Related U.S. Application Data
(60) Provisional application No. 61/525,367, filed on Aug. 19, 2011.

(30) Foreign Application Priority Data
Jul. 14, 2011 (EP) 11382238.1

(51) Int. Cl. C12Q 1/68

(52) U.S. Cl. CPC C12Q 1/6886; C12Q 2600/16; C12Q 2600/158; C12Q 2600/118
USPC 506/9; 506/16; 435/6.12; 506/7

(57) ABSTRACT

The invention relates to a method for predicting the outcome of a subject suffering from cancer, based on the copy number of the CHKA gene in a sample from said subject. The invention also relates to a BAC composition and a method for detecting the number of copies of the CHKA gene.

### FIELD OF THE INVENTION

The invention relates to the field of diagnosis and, more in particular, to a method for diagnosing or for predicting the outcome of a subject suffering from cancer, based on the copy number of the choline kinase alpha (CHKA) gene in a sample from said subject. The invention also relates to a bacterial artificial chromosome (BAC) composition and a method for detecting the number of copies of the CHKA gene.

### BACKGROUND OF THE INVENTION

Cancer is a term used for diseases in which abnormal cells divide without control and are able to invade other tissues. Cancer cells can spread to other parts of the body through the blood and lymph systems. Cancer is not just one disease but many diseases.

There are more than 100 different types of cancer. Most cancers are named for the organ or type of cell in which they start, for example, cancer that begins in the colon is called colon cancer; cancer that begins in lung is called lung cancer.

Rates of new diagnoses and rates of death from all cancers combined declined significantly in the most recent time period for men and women overall and for most racial and ethnic populations in the United States, according to a report from leading health and cancer organizations.

The drops are driven largely by declines in rates of new cases and rates of death for the three most common cancers in men (lung, prostate, and colorectal cancers) and for two of the three leading cancers in women (breast and colorectal cancer). New diagnoses for all types of cancer combined in the United States decreased, on average, almost 1% per year from 1999 to 2006. Cancer deaths decreased 1.6% per year from 2001 to 2006 (Kohler B A et al. Report to the Nation on the Status of Cancer, 1975-2007, Featuring Tumors of the Brain and Other Nervous System. JNCI; May 4, 2011).

Lung cancer is one of the leading causes of worldwide death, and non-small cell lung cancer (NSCLC) accounts for approximately 85% of all lung cancers, with 1.2 million new cases worldwide each year. NSCLC resulted in more than one million deaths worldwide in 2001 and is the leading cause of cancer-related mortality in both men and women (31% and 25%, respectively).

NSCLC comprises a group of heterogeneous diseases grouped together because their prognosis and management is roughly identical. However, the following subtypes based on their histology can be identified: (i) squamous cell carcinoma (SCC), accounting for 30% to 40% of NSCLC, starts in the larger breathing tubes but grows slower meaning that the size of these tumours varies on diagnosis, (ii) adenocarcinoma is the most common subtype of NSCLC, accounting for 50% to 60% of NSCLC, which starts near the gas-exchanging surface of the lung and which includes a subtype, the bronchioalveolar carcinoma, which may have different responses to treatment and (iii) large cell carcinoma is a fast-growing form that grows near the surface of the lung. It is primarily a diagnosis of exclusion, and when more investigation is done, it is usually reclassified to squamous cell carcinoma or adenocarcinoma.

A common characteristic of human tumour cell lines is the high basal levels of phosphocholine (PCho) as consequence of CHKA overexpression. This parameter is able to distinguish between normal and tumour cell lines, independently of their proliferative status. These results have been confirmed in different human tumours, including breast, lung, colon, bladder, prostate tumours (Ramirez de Molina A et al. Oncogene 21: 4317-4322, 2002), brain (Nelson S J et al. Neoplasia 2: 166-189, 2000) and ovary (Ramoni C, et al. Cancer Res. 65: 9369-9376, 2005). In all of them, high levels of ChoK activity and PCho can be observed. These biochemical discoveries suggest that CHKA could be a potential tumour marker in a wide variety of human cancers. Moreover, it has recently been demonstrated that CHKA over expression in the human cell line HMEC (Human Mammary Epithelial Cells) provokes an induction of DNA synthesis and also that breast cancer cells need ChoK activity for their proliferation, supporting a strong link between CHKA and tumour progression (Ramirez de Molina et al. 2004. Cancer Research 64, 6732-6739). Around 50% of lung, colon and prostate tumours show high levels of CHKA. Only 18% of breast cancer tumours show high level of this enzyme; however 38% of these samples present increased enzymatic activity with a mean of 10 fold increase compared to normal tissue.

Gene amplification is a common mechanism in deregulated oncogene expression, mainly in solid tumours. An example is the tyrosine kinase membrane receptors which regulate signalling pathways of proliferation and apoptosis. Different alterations in the genes which encode these receptors are targets of new therapeutic strategies based on blocking the receptor or the inhibition of its tyrosine kinase activity. Another example is the oncogene HER2 in breast cancer where the copy number amplification is associated to poor outcome and has predictive value for sensitivity and resistance to therapy. This finding has led to the inclusion of the HER2 gene in the clinical panels as a predictive factor of breast cancer (Carlsson et al. Br J Cancer. 2004, 90: 2344-8, Villman et al. Acta Oncol. 2006; 45: 590-6). Another example is the EGFR (Epidermal growth factor receptor) gene in lung cancer. Although common gene alterations consist in small deletions or insertions or point mutations, recent studies have correlated other gene alterations as amplification or EGFR polysomy with the tumour aggressiveness and response of gefitinib (Dacic et al. Am J Clin Pathol. 2006, 125: 860-5). Thus, there is a need in the art for further diagnosis methods for cancer and prognosis methods for predicting the clinical outcome of a patient suffering from cancer, and specifically from NSCLC.

### SUMMARY

In one aspect, the invention relates to a composition comprising at least two polynucleotides selected from the group consisting of:
(a) a polynucleotide comprising the human genomic fragment present in the CTD-2162L23 BAC (SEQ ID NO:1) or a variant thereof which hybridizes under stringent conditions to said polynucleotide,
(b) a polynucleotide comprising the human genomic fragment present in the RP11-314K20 BAC (SEQ ID NO:2) or a variant thereof which hybridizes under stringent conditions to said polynucleotide,
(c) a polynucleotide comprising the human genomic fragment present in the CTD-2655K5 BAC (SEQ ID NO:3) or a variant thereof which hybridizes under stringent conditions to said polynucleotide and
(d) a polynucleotide comprising the human genomic fragment present in CTC-783C1 BAC (SEQ ID NO:4) or a variant thereof which hybridizes under stringent conditions to said polynucleotide.

In another aspect, the invention relates to a method for determining the copy number of the CHKA gene which comprises:
(a) contacting a sample containing genetic material with a composition according to any of claims 1 to 3 under conditions adequate for the hybridisation of the polynucleotides forming part of the composition with the CHKA gene or genes present in the sample; and
(b) determining the copy number of the CHKA gene based on the hybridisation of the composition with the sample containing genetic material.

In yet another aspect, the invention relates to an in vitro method for predicting the outcome of a subject suffering from cancer comprising detecting whether the CHKA gene is amplified in a sample from said subject, wherein amplification of the CHKA gene is indicative of an unfavourable outcome of the subject.

### BRIEF DESCRIPTION OF THE FIGURES

FIG. 1 shows Kaplan-Meier plots for CHKA copy number and overall survival (A) and CHKA copy number and disease-free survival (B).

### DETAILED DESCRIPTION OF THE INVENTION

The inventors of the present invention have found that, surprisingly, the copy number of the CHKA gene can be determined by FISH (fluorescent in situ hybridization) using a specific polynucleotide composition. Moreover, the authors of the present invention have also found out that there is a statistically significant correlation between the copy number of the CHKA gene and the prognosis of cancer patient and, in particular, in patients suffering from non-small cell lung cancer (NSCLC). Thus, the determination of the copy number of the CHKA gene is useful for determining the prognosis of a subject suffering from cancer. In this sense, an increased copy number of the CHKA gene correlates with an unfavourable prognosis of the subject suffering from cancer. Based on these findings, the inventors have developed the methods of the present invention in their different embodiments that will be described now in detail.

Compositions and Kit-of-Parts of the Invention

The authors have successfully identified a plurality of genomic fragments derived from human chromosome 11q13 that, when used in combination, allow the detection of the amplification of the CHKA gene. Thus, in a first aspect, the invention relates to a composition, hereinafter composition of the invention, comprising at least two polynucleotides selected from the group consisting of:
(a) a polynucleotide comprising the human genomic fragment present in the CTD-2162L23 BAC or a variant thereof which hybridizes under stringent conditions to said polynucleotide,
(b) a polynucleotide comprising the human genomic fragment present in the RP11-314K20 BAC or a variant thereof which hybridizes under stringent conditions to said polynucleotide,
(c) a polynucleotide comprising the human genomic fragment present in the CTD-2655K5 BAC or a variant thereof which hybridizes under stringent conditions to said polynucleotide and
(d) a polynucleotide comprising the human genomic fragment present in CTC-783C1 BAC or a variant thereof which hybridizes under stringent conditions to said polynucleotide.

The term “composition”, as used herein, refers to any mixture of two or more polynucleotides according to the invention. It can be a solution, a suspension, an emulsion, liquid, powder, a paste, aqueous, non-aqueous or any combination of such ingredients. The term “composition” has to be understood also as a kit of parts wherein the two or more components of the kit of parts are physically separated. The parts of the kit of parts can then, e.g., be used simultaneously or chronologically staggered, that is at different time points and with equal or different time intervals for any part of the kit of parts. The ratio of the total amounts of the components of the kit to each other being used in the combined preparation/composition can be varied, e.g. in order to cope with the needs of a diagnostic procedure. Additionally, the kit-of-parts according to the invention can contain instructions for the simultaneous, sequential or separate use of the components in the kit. Said instructions can be in the form of printed material or in the form of an electronic support, capable of storing the instructions such that they can be read by a subject, such as electronic storage media (magnetic disks, tapes and similar), optical media (CD-ROM, DVD) and the like. Additionally or alternatively, the media can contain Internet addresses providing said instructions.

The BACs are hereinafter referred using the NCBI-recommended clone nomenclature. For instance, the term “CTD-2162L23” indicates a clone in the “CTD” BAC library. More precisely, it is a clone found in the microtiter dish “2162” at the intersection of row “L” and column “23”.

The term “CTD-2162L23” corresponds to a BAC which comprises a region of chromosome 11 having a length of 126394 by and starting at position 67821470 and ending at position 67947863 according to the sequence of chromosome 11 in the UCSC Human Genome Browser (GRCh37/hg19 Assembly of Feb. 2009). Its nucleotide sequence is shown in SEQ ID NO:1.

The term “RP11-314K20” corresponds to a BAC which comprises a region of chromosome 11 having a length of 192918 by and starting at position 67716432 and ending at position 67909349 according to the sequence of chromosome 11 in the UCSC Human Genome Browser (GRCh37/hg19 Assembly of Feb. 2009). Its nucleotide sequence is shown in SEQ ID NO:2.

The term “CTD-2655K5” corresponds to a BAC which comprises a region of chromosome 11 having a length of 191582 by and starting at position 67839236 and ending at position 68030817 according to the sequence of chromosome 11 in the UCSC Human Genome Browser (GRCh37/hg19 Assembly of February 2009). Its nucleotide sequence is shown in SEQ ID NO:3.

The term “CTC-783C1” corresponds to a BAC which comprises a region of chromosome 11 having a length of 154055 by and starting at position 67800991 and ending at position 67955045 according to the sequence of chromosome 11 in the UCSC

Human Genome Browser (GRCh37/hg19 Assembly of February 2009). Its nucleotide sequence is shown in SEQ ID NO:4.

The term “polynucleotide”, as used herein, shall mean any single or double stranded nucleotide polymer of genomic origin or some combination thereof, which by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence.

The term “human genomic fragment”, as used in the present invention refers to a specific fragment comprising a gene or a fragment of a gene of human origin or a variant thereof. A person skilled in the art will understand that the present invention relates not only to the genomic fragments mentioned in the present invention, but also to variants thereof. A person skilled in the art will also understand that the variant of the human genomic fragment can be of any mammal, such as simians, mice, rats, pigs, dogs, rabbits, cats, cows, horses and goats.

The term “variant of the human genomic fragment” according to the present invention relates to any polynucleotide in which one or more nucleotides of the polynucleotides according to the invention are deleted, added or substituted. As is known in the state of the art, the identity between two nucleotide sequences is determined by comparing the sequence of a first and a second nucleic acid. Variants according to the present invention include nucleic acid sequences presenting at least 60%, 65%, 70%, 72%, 74%, 76%, 78%, 80%, 90% or 95% similarity or identity with the original nucleotide sequence. The degree of identity between two nucleic acids is determined by using computational algorithms and methods which are widely known by the persons skilled in the art. The identity between two nucleic acid sequences will preferably be determined by using the BLAST algorithm [BLAST Manual, Altschul, S., et al., NCBI NLM NIH Bethesda, Md. 20894, Altschul, S., et al., J. Mol. Biol. 215: 403-410 (1990)].

The variant of the human genomic fragments include fragments which preserve the capacity to hybridize under stringent conditions to the polynucleotides comprising the human genomic fragments present in the BACs as mentioned above.

“Stringent hybridisation” is used herein to refer to conditions under which nucleic acid hybrids are stable. As known to those of skill in the art, the stability of hybrids is reflected in the melting temperature (Tm) of the hybrids. In general, the stability of a hybrid is a function of ionic strength, temperature, G/C content, and the presence of chaotropic agents. The Tm values for polynucleotides can be calculated using known methods for predicting melting temperatures (see, e.g., Baldino et al, Methods Enzymology 168:761-777; Bolton et al, 1962, Proc. Natl. Acad. Sci. USA 48: 1390; Bresslauer et al, 1986, Proc. Natl. Acad. Sci USA 83:8893-8897; Freier et al, 1986, Proc. Natl. Acad. Sci USA 83:9373-9377; Kierzek et al, Biochemistry 25:7840-7846; Rychlik et al, 1990, Nucleic Acids Res 18:6409-6412 (erratum, 1991, Nucleic Acids Res 19:698; Sambmok et al, ad supra); Suggs et al, 1981, In Developmental Biology Using Purified Genes (Brown et al, eds.), pp. 683-693, Academic Press; and Wetmur, 1991, Crit Rev Biochem MoI Biol 26:227-259. All publications are incorporated herein by reference). In some embodiments, the polynucleotide encodes the polypeptide disclosed herein and hybridizes under defined conditions, such as moderately stringent or highly stringent conditions, to the complement of a sequence comprising the genomic fragment present in the BACs as defined above.

The term “moderately stringent conditions” as used herein refers to conditions that permit target-DNA to bind a complementary nucleic acid that has about 60 percent identity, preferably about 75 percent identity, about 85 percent identity to the target DNA, with greater than about 90 percent identity to target-polynucleotide. Exemplary moderately stringent conditions are conditions equivalent to hybridisation in 50% formamide, 5× Denhardt's solution, 5×SSPE, 0.2% SDS at 42° C., followed by washing in 0.2×SSPE, 0.2% SDS, at 42° C.

The term “highly stringent conditions”, as used herein refers generally to conditions that are about 10 degrees centigrade or less from the thermal melting temperature Tm as determined under the solution condition for a defined polynucleotide sequence. In some embodiments, a high stringency condition refers to conditions that permit hybridisation of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). Exemplary high stringency conditions can be provided, for example, by hybridisation in conditions equivalent to 50% formamide, 5× Denhardt's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. degrees centigrade and washing in 0.1×SSC containing 0.1% SDS at 65° C.

Other high stringency hybridisation conditions, as well as moderately stringent conditions, are described in the references cited above.

In a preferred embodiment, the human genomic fragments are found within cloning vehicles in order to allow their propagation. The skilled person will understand that the cloning vehicles are those which can accommodate human genomic fragments having the size of the genomic fragments according to the invention. Thus, preferably, the cloning vehicle is an artificial chromosome. The cloning vehicle can comprise an artificial chromosome comprising a bacterial artificial chromosome (BAC), a bacteriophage P1-derived vector (PAC), a yeast artificial chromosome (YAC), or a mammalian artificial chromosome (MAC). In a preferred embodiment, the human genomic fragments are provided in BACs.

The term “bacterial artificial chromosome” (BAC), as used herein, refers to a DNA construct, based on a functional fertility plasmid (or F-plasmid), which has a low copy number because of the strict control of replication, used for transforming and cloning in bacteria, usually E. coli. The BAC's usual insert size is 150-350 kbp in which medium-sized segments of DNA (100,000 to 300,000 bases in length) that come from another species are cloned into bacteria. Once the foreign DNA has been cloned into the bacteria's chromosome, many copies of it can be made (amplified) and sequenced. In addition, BAC libraries of human genomic DNA have more complete and accurate representation of the human genome than libraries in cosmids or yeast artificial chromosomes. BACs are described in further detail in U.S. application Ser. Nos. 10/659,034 and 61/012,701, which are hereby incorporated by reference in their entireties.

The present invention comprises any combination of two or more of the polynucleotides (a) to (d) as defined above. Thus, in preferred embodiment, the composition according to the invention comprises the following combinations of polynucleotides: (a) and (b); (a) and (c), (a) and (d); (b) and (c); (b) and (d); (c) and (d); (a), (b) and (c); (a), (b) and (d); (a), (c) and (d); (b), (c) and (d) and (a), (b), (c) and (d). In a preferred embodiment, the composition comprises the polynucleotides (a), (b), (c) and (d). In a preferred embodiment, the BACs are the CTD-2162L23, RP11-314K20, CTD-2655K5 and CTC-783C1 BACs, already defined above. The BACs are commercially available, for example, the RP11 BAC library is available from the BAC Resources PAC (Children's Hospital Oakland Research Institute), from Empire Genomics, imaGenes or Invitrogen; and the CTD and CTC BAC libraries are available from Thermo Fisher Scientific Inc.

Method For Determining the Copy Number of the CHKA Gene

In another aspect, the invention relates to a method for determining the copy number of the CHKA gene, hereinafter referred to as the first method of the invention, which comprises:
(a) contacting a sample containing genetic material with the composition of the invention under conditions adequate for the hybridisation of the composition with the CHKA gene or genes present in the sample; and
(b) determining the copy number of the CHKA gene based on the hybridisation of the composition with the sample containing genetic material.

The CHKA gene, as used in the present invention, refers to the gene which codes for the enzyme Choline kinase alpha (CHKA), that catalyzes the chemical reaction between ATP and choline to make a phosphocholine (EC 2.7.1.32). The skilled person in the art will understand that it is possible to use the first method of the invention in CHKA from different species. Thus, the invention considers the determination of the copy number of the CHKA gene from human origin, as defined in the NCBI database, version Mar. 29, 2011 (positions 67820326 to 67888858 in the genomic region shown in the NCBI database under accession number NC_{—}000011.9), but also of CHKA from rat (Rattus norvegicus) (positions 206368963 to 206418526 in the genomic region shown in the NCBI database under accession number NC_{—}005100.2), from mouse (Mus musculus) (positions 3851773 to 3894367 in the genomic region shown in the NCBI database under accession number NC_{—}000085.5), from bull (Bos Taurus) (positions 47653321 to 47685935 in the genomic region shown in the NCBI database under accession number NC_{—}007330.4), from Danio rerio (positions 20903529 to 20926085 in the genomic region shown in the NCBI database under accession number NC_{—}007129.5), from Gallus gallus (positions 17301687 to17319617 in the NCBI database under accession number NC_{—}006092.2), from Canis lupus familiaris (positions 52771483 to 52807744 in the NCBI database under accession number NC_{—}006600.2), from common chimpanzee (Pan troglodytes) (positions 65735780 to 65807409 in the NCBI database under accession number NC_{—}006478.3), etc.

In a first step, the first method of the invention comprises contacting a sample containing genetic material with a composition according to the invention under conditions adequate for the hybridisation of the polynucleotides forming part of the composition with the CHKA gene or genes present in the sample.

The term “sample”, as used herein, relates to any sample which can be obtained from the subject and which contains genetic material. The present method can be applied to any kind of biological sample from a subject, such as a biopsy sample, tissue, cell or fluid (serum, saliva, semen, sputum, cerebral spinal fluid (CSF), tears, mucus, sweat, milk, brain extracts and the like). Methods of obtaining a biological sample from a subject are known in the art. Exemplary biological samples may be isolated from normal cells or tissues, or from neoplastic cells or tissues. Neoplasia is a biological condition in which one or more cells have undergone characteristic anaplasia with loss of differentiation, increased rate of growth, invasion of surrounding tissue, and which cells may be capable of metastasis. In particular examples, a biological sample includes a tumour sample, such as a sample containing neoplastic cells.

The samples described herein can be prepared using any method now known or hereafter developed in the art. Generally, tissue samples are prepared by fixing and embedding the tissue in a medium (such as paraffin). In other examples, samples include a cell suspension which is prepared as a monolayer on a solid support (such as a glass slide) for example by smearing or centrifuging cells onto the solid support. In further examples, fresh frozen (for example, unfixed) tissue sections may be used in the methods disclosed herein.

In some examples an embedding medium is used. An embedding medium is an inert material in which tissues and/or cells are embedded to help preserve them for future analysis. Embedding also enables tissue samples to be sliced into thin sections. Embedding media include paraffin, celloidin, OCT™ compound, agar, plastics, or acrylics.

Many embedding media are hydrophobic; therefore, the inert material may need to be removed prior to histological or cytological analysis, which utilizes primarily hydrophilic reagents. The term deparaffinization or dewaxing is broadly used herein to refer to the partial or complete removal of any type of embedding medium from a biological sample. For example, paraffin-embedded tissue sections are dewaxed by passage through organic solvents, such as toluene, xylene, limonene, or other suitable solvents. The process of fixing a sample can vary. Fixing a tissue sample preserves cells and tissue constituents in as close to a life-like state as possible and allows them to undergo preparative procedures without significant change. Fixation arrests the autolysis and bacterial decomposition processes that begin upon cell death, and stabilizes the cellular and tissue constituents so that they withstand the subsequent stages of tissue processing, such as for in situ hybridisation (ISH).

Tissues can be fixed by any suitable process, including perfusion or by submersion in a fixative. Fixatives can be classified as cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), fixatives of unknown mechanism (e.g., mercuric chloride, acetone, and picric acid), combination reagents (e.g., Carnoy's fixative, methacarn, Bouin's fluid, B5 fixative, Rossman's fluid, and Gendre's fluid), microwaves, and miscellaneous fixatives (e.g., excluded volume fixation and vapor fixation). Additives may also be included in the fixative, such as buffers, detergents, tannic acid, phenol, metal salts (such as zinc chloride, zinc sulfate, and lithium salts), and lanthanum.

The most commonly used fixative in preparing samples for immunohistochemistry (IHC) is formaldehyde, generally in the form of a formalin solution (4 percent formaldehyde in a buffer solution, referred to as 10 percent buffered formalin). In one example, the fixative is 10 percent neutral buffered formalin.

The expression “conditions adequate for the hybridisation” have been described in detail above in the context of the compositions of the invention. Although the conditions may vary depending on the nature of the material under study, these conditions can be determined by the skilled person using routine procedures. The conditions are preferably those allowing hybridisation under moderately stringent conditions or under high stringent conditions. The “conditions adequate for the hybridisation” will be adjusted by a skilled person in the art and will vary depending upon the nature of the hybridisation method and the composition and length of the hybridizing nucleic acid sequences of the composition of the invention. Generally, the temperature of hybridisation and the ionic strength (such as the sodium concentration) of the hybridisation buffer will determine the stringency of hybridisation. Calculations regarding hybridisation conditions for attaining particular degrees of stringency are discussed in Sambrook et al., (1989) Molecular Cloning, second edition, Cold Spring Harbor Laboratory, Plainview, N.Y. (chapters 9 and 11) and in Ausubel et al., Short Protocols in Molecular Biology: A Compendium of Methods from Current Protocols in Molecular Biology, 4th ed., Wiley and Sons, 1999.

The second step of the first method of the invention involves determining the copy number of the CHKA gene based on the hybridisation of the composition with the sample containing genetic material.

The term “copy number”, as used herein, refers to the number of copies of a nucleic acid molecule in a cell. The copy number includes the number of copies of one or more genes or portions thereof in genomic (chromosomal) DNA of a cell. In a normal cell (such as a non-tumour cell), the copy number of a gene (or any genomic DNA) is usually about two (one copy on each member of a chromosome pair). In some examples, the copy number of a gene or nucleic acid molecule includes an average copy number taken from a population of cells.

Methods of determining the copy number of a gene or chromosomal region are well known to those of skill in the art. Said methods include, without limitation, in situ hybridisation (such as fluorescent, chromogenic, or silver in situ hybridisation), comparative genomic hybridisation, or polymerase chain reaction (such as real-time quantitative PCR). In a particular embodiment, the copy number of the CHKA gene is determined by in situ hybridisation (ISH), such as fluorescence in situ hybridisation (FISH), chromogenic in situ hybridisation (CISH), or silver in situ hybridisation (SISH). In a preferred embodiment, the copy number of the CHKA gene is determined by FISH.

The term “fluorescent in situ hybridisation” or FISH, as used in the present invention, refers to a cytogenetic technique that is used to detect and localize the presence or absence of specific DNA sequences on chromosomes. FISH uses fluorescent probes that bind to only those parts of the chromosome with which they show a high degree of sequence similarity. As the probe for FISH, a DNA fragment, a PCR product, a cDNA, a PAC clone or a BAC clone (each of which has a sequence of interest) may be used. In a typical method using FISH, a DNA probe (a CHKA probe) is labeled with a fluorescent dye or a hapten, typically in the form of fluor-dUTP, digoxigenin-dUTP, biotin-dUTP or hapten-dUTP that is incorporated into the DNA using enzymatic reactions, such as nick translation or PCR. The sample containing the genetic material (chromosome samples) may be smears on slide glass prepared from cultured cells of an isolated cancer. Alternatively, chromosome samples may be slide samples sliced from formalin-fixed, paraffin-embedded cancer-containing tissue blocks. After hardening for prevention of falling off from the slide glass during hybridisation process, chromosome sample slides are denatured by formamide treatment. The labeled probe is then hybridized to the sample containing genetic material on slides under appropriate conditions, which will be determined by the skilled person in the art. After hybridisation, the labeled sample is visualized either directly (in the case of a fluor-labeled probe) or indirectly (using fluorescently labeled antibodies to detect a hapten-labeled probe).

In the case of CISH, the probe is labeled with digoxigenin, biotin, or fluorescein and hybridised to the sample containing genetic material (chromosome or nuclear preparations) under appropriate conditions. The probe is detected with an anti-digoxigenin, -biotin or -fluorescein antibody, which is either conjugated to an enzyme (such as horseradish peroxidase or alkaline phosphatase) that produces a coloured product at the site of the hybridized probe in the presence of an appropriate substrate (such as DAB, NBT/BCIP, etc.), or with a secondary antibody conjugated to the enzyme. SISH is similar to CISH, except that the enzyme (such as horseradish peroxidase) conjugated to the antibody (either anti-hapten antibody or a secondary antibody) catalyzes deposition of metal nanoparticles (such as silver or gold) at the site of the hybridized probe. For any ISH method, CHKA gene copy number may be determined by counting the number of fluorescent, coloured, or silver spots on the chromosome or nucleus.

Any label that can be attached to a nucleic acid molecule (such as CHKA nucleic acid) can be used to label the CHKA probes, thereby permitting detection of the nucleic acid molecule. Examples of labels include, but are not limited to, radioactive isotopes, enzyme substrates, co-factors, ligands, chemiluminescent agents, fluorophores, haptens, enzymes, and combinations thereof. Methods for labelling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley and Sons, New York, 1998).

In a particular embodiment, the contacting step is carried out using a composition comprising polynucleotides comprising the human genomic fragment present in the CTD-2162L23, RP11-314K20, CTD-2655K5 and CTC-783C1 BACs. The BACs have been defined above.

The preferred method of the first method of the invention includes the following steps:
(a) The BACs are transformed into a bacteria (as E.coli) and the bacteria culture is grown in an appropriate medium and the BACs DNA extracted;
(b) Labelling of the DNA extracted from the BACs using the “nick translation” technique, wherein the DNA is treated with DNase to produce single-stranded “nicks”. This is followed by replacement in nicked sites by DNA polymerase I, which elongates the 3′ hydroxyl terminus, removing nucleotides by 5′-3′ exonuclease activity, replacing them with dNTPs. Then a fluorophore is attached for fluorescent labelling, or an antigen for immunodetection (e.g. digoxigenin or biotin). When DNA polymerase I eventually detaches from the DNA, it leaves another nick in the phosphate backbone. The nick has “translated” some distance depending on the processivity of the polymerase. This nick could be sealed by DNA ligase, or its 3′ hydroxyl group could serve as the template for further DNA polymerase I activity. Proprietary enzyme mixes are available commercially to perform all steps in the procedure in a single incubation, for example the commercial kit of Vysis (Downers Grove, USA).
(c) Denaturation of the labeled DNA extracted from the BAC, preferably at 75° C.
(d) Cells with the sample containing the genetic material are fixed on slides and a denaturation step is performed with formamide and a temperature preferably of 42° C.
(e) Perform the contacting step or hybridisation between the sample containing the genetic material and the labeled DNA extracted from the BAC in the denaturated form, as explained previously.
(f) Addition of antibodies anti-digoxigenin or avidin linked with a fluorophore.
(g) Detection of the signal by microscopy and determination of the copy number of the CHKA gene.

Method For Predicting the Outcome of a Patient Suffering Cancer

Moreover, the authors of the present invention have observed that amplification of the CHKA gene is indicative of a poor outcome of patients diagnosed with cancer. For instance, the example of the present invention describes that patients comprising more than 4 copies of the CHKA gene show reduced survival and relapse-free survival than patients wherein the number of copies of the CHKA gene is reduced. Thus, in another aspect, the invention relates to an in vitro method, hereinafter second method of the invention, for predicting the outcome of a subject suffering from cancer comprising detecting whether the CHKA gene wherein is amplified in a sample from said subject, wherein amplification of the CHKA gene is indicative of an unfavourable outcome of the subject.

The term “predicting the outcome”, as used herein, refers to a medical term to describe the likely outcome of an illness or progression of the course of a disease or to the prognosis of a disease, such as cancer (for example, non-small cell lung cancer). The prediction or prognosis can include determining the likelihood of a subject to develop aggressive, recurrent disease, to develop one or more metastases, to survive a particular amount of time (e.g., determine the likelihood that a subject will survive 1, 2, 3, 4, or 5 years), to survive a particular amount of time without disease progression (e.g., determine the likelihood that a subject will survive 1, 2, 3, 4, or 5 years without progression), to respond to a particular therapy (e.g., chemotherapy), or combinations thereof.

As will be understood by those skilled in the art, the prognostic methods according to the present invention, although preferred to be, need not be correct for 100% of the subjects to be diagnosed or evaluated. The term, however, requires that a statistically significant portion of subjects can be identified as having increased probability of having a given outcome. Whether a subject is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% at least 95%. The p-values are, preferably, 0.05, 0.025, 0.01, 0.001 or lower.

Any parameter which is widely accepted for determining the progression of a patient can be used for predicting the outcome of a subject and include, without limitation:
disease-free progression which, as used herein, describes the proportion of subjects in complete remission who have had no recurrence of disease during the time period under study.
disease-free survival (DFS), as used herewith, is understood as the length of time after treatment for a disease during which a subject survives with no sign of the disease.
objective response which, as used in the present invention, describes the proportion of treated subjects in whom a complete or partial response is observed.
tumour control which, as used in the present invention, relates to the proportion of treated subjects in whom complete response, partial response, minor response or stable disease ≧6 months is observed.
progression free survival which, as used herein, is defined as the time from start of treatment to the first measurement of cancer growth.
Time to progression (TTP), as used herein, relates to the time after a disease is treated until the disease starts to get worse. The term “progression” has been previously defined.
six-month progression free survival or “PFS6” rate which, as used herein, relates to the percentage of subjects wherein free of progression in the first six months after the initiation of the therapy and
median survival which, as used herein, relates to the time at which half of the subjects enrolled in the study are still alive.

The term “subject”, as used herein, refers to all animals classified as mammals and includes, but is not restricted to, domestic and farm animals, primates and humans, e.g., human beings, non-human primates, cows, horses, pigs, sheep, goats, dogs, cats or rodents. Preferably, the subject is a male or female human of any age or race. In the context of the present invention, the subject is a subject suffering from cancer or previously diagnosed with cancer.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer. In a preferred embodiment, the cancer is selected from the group of breast cancer, colon cancer, lung cancer, bladder cancer and prostate cancer.

The term “lung cancer” is meant to refer to any cancer of the lung and includes non-small cell lung carcinomas and small cell lung carcinomas. In a preferred embodiment, the second method of the invention is applicable to a subject suffering from NSCLC. In a particular embodiment, the NSCLC is selected from squamous cell carcinoma of the lung, large cell carcinoma of the lung, and adenocarcinoma of the lung. Furthermore, the present method can also be applicable to a subject suffering from any stage of NSCLC (stages 0, IA, IB, IIa, IIb, IIIa, IIIb or IV). In a preferred embodiment, the stage of NSCLC is advanced NSCLC, preferably, stages IIIa, IIIb or IV.

The terms “gene amplification” and “gene duplication” (and variants such as “amplification of a gene” or “duplication of a gene”) are used interchangeably and refer to a process by which multiple copies of a gene or gene fragment are formed in a particular cell or cell line. The copies of the gene are not necessarily located in the same chromosome. The duplicated region (a stretch of amplified DNA) is often referred to as an “amplicon”. Usually, the amount of the messenger RNA (mRNA) produced, i.e., the level of gene expression, also increases in proportion to the number of copies made of the particular gene.

The term “sample”, as used herein, relates to any sample which can be obtained from the subject. The present method can be applied to any kind of biological sample from a subject, such as a biopsy sample, tissue, cell or fluid (serum, saliva, semen, sputum, cerebral spinal fluid (CSF), tears, mucus, sweat, milk, brain extracts and the like). In a particular embodiment, said sample is a tissue sample, preferably a tumour tissue sample, more preferably a lung tumour tissue sample from a subject suffering from NSCLC. Said sample can be obtained by conventional methods, e.g., biopsy, by using methods well known to those of ordinary skill in the related medical arts. Methods for obtaining the sample from the biopsy include gross apportioning of a mass, or microdissection or other art-known cell-separation methods. Tumour cells can additionally be obtained from fine needle aspiration cytology. In order to simplify conservation and handling of the samples, these can be formalin-fixed and paraffin-embedded or first frozen and then embedded in a cryosolidifiable medium, such as OCT-Compound, through immersion in a highly cryogenic medium that allows for rapid freeze.

In a particular embodiment, the detection of the amplification of the CHKA gene comprises determining the copy number of the CHKA gene. Methods of determining the copy number of the CHKA gene are well known to those of skill in the art. The methods include, without limitation, in situ hybridisation (such as fluorescent in situ hybridisation (FISH), chromogenic, or silver in situ hybridisation), comparative genomic hybridisation, or polymerase chain reaction (such as real-time quantitative PCR). See for example, Avison, M., Measuring Gene Expression, New York: Taylor and Francis Group, 2007, Allison, D. B., et al, ed. DNA Microarrays and Related Genomics Techniques: Design, Analysis, and Interpretation of Experiments (Biostatistics), Boca Raton: Chapman and Hill/CRC, 2006; Hayat M. A., ed., Handbook of Immunohistochemistry and in situ Hybridisation of Human Carcinomas, Burlington: Elsevier Academic Press, 2004.

In another example, CHKA gene copy number is determined by comparative genomic hybridisation (CGH). See, e.g., Kallioniemi et al, Science 258:818-821, 1992; U.S. Pat. Nos. 5,665,549 and 5,721,098. In one example, CGH includes the following steps. DNA from tumour tissue (such as a lung cancer sample) and from normal control tissue (reference, such as a non-tumour sample) is labeled with different detectable labels, such as two different fluorophores. After mixing tumour and reference DNA along with unlabeled human Cot-1 DNA (placental DNA that is enriched for repetitive DNA sequences such as the Alu and Kpn family) to suppress repetitive DNA sequences, the mix is hybridized to normal metaphase chromosomes. The fluorescence intensity ratio along the chromosomes is used to evaluate regions of DNA gain or loss in the tumour sample. In a further example, CHKA gene copy number is determined by array CGH (aCGH). See, e.g., Pinkel and Albertson, Nat. Genet. 37:S11-S17, 2005; Pinkel et al, Nat. Genet. 20:207-211, 1998; Pollack et al, Nat. Genet. 23:41-46, 1999. Similar to standard CGH, tumour and reference DNA are differentially labeled and mixed. However, for aCGH, the DNA mixture is hybridized to a slide containing hundreds or thousands of defined DNA probes (such as probes that are homologous to portions of the CHKA gene). The fluorescence intensity ratio at each probe in the array is used to evaluate regions of DNA gain or loss in the tumour sample, which can be mapped in finer detail than CGH, based on the particular probes which exhibit altered fluorescence intensity. In one example, the array is an Agilent Human Genome CGH 44B Oligo Microarray (Agilent Technologies, Santa Clara, Calif.). In another example, the CGH array is a Whole Genome Tiling, Custom, or Chromosome specific Tiling Array (for example, a Chromosome 15 Tiling Array) as provided by Roche NimbleGen, Inc. (Madison, Wis.).

In general, CGH (and aCGH) does not provide information as to the exact number of copies of a particular genomic DNA or chromosomal region. Instead, CGH provides information on the relative copy number of one sample (such as a tumour sample, for example a lung cancer sample) compared to another (such as a control sample, for example a non-tumour cell or tissue sample). Thus, CGH is most useful to determine whether CHKA gene copy number of a sample is increased or decreased as compared to a control sample (such as a non-tumour cell or tissue sample or a reference value).

Additional methods that may be used to determine copy number of the insulin-like growth factor 1 receptor (IGF1R) gene are known to those of skill in the art. Such methods include, but are not limited to Southern blotting, multiplex ligation-dependent probe amplification (MLPA; see, e.g., Schouten et al., Nucl. Acids Res. 30:e57, 2002), and high-density SNP genotyping arrays (see, e.g. WO 98/030883).

Also disclosed herein is a method of scoring (for example, enumerating) copy number of a gene in a sample from a subject (such as a subject with neoplastic disease), wherein the sample is stained by ISH (such as FISH, SISH, CISH, or a combination of two or more thereof) for the gene of interest and wherein individual copies of the gene are distinguishable in cells in the sample. Typically, the method includes identifying individual cells in a sample with the highest number of signals per nucleus for the gene (such as the strongest signal in the sample), counting the number of signals for the gene in the identified cells, and determining an average number of signals per cell, thereby scoring the gene copy number in the sample. In additional embodiments, the method further includes counting the number of signals for a reference (such as a chromosomal locus known not to be abnormal, for example, centromeric DNA) and determining an average ratio of the number of signals for the gene to the number of signals for the reference per cell.

The scoring method includes identifying individual cells in the sample (such as a tissue section or tumour core) having the highest number of signals (such as the highest number of spots per cell or the brightest intensity of staining) for the gene of interest in the cells in the sample. Thus, the disclosed method does not determine gene copy number in a random sampling of cells in the sample. Rather, the method includes specifically counting gene copy number in those cells that have the highest gene copy number in the sample. In some examples, identifying the individual cells having the highest number of signals for the gene includes examining a sample stained by ISH for the gene under low power microscopy (such as about 20× magnification). Cells with the strongest signal (for example, highest amplification signal under higher power) are identified for counting by eye or by an automated imaging system. In some examples, such as when the sample is a tissue section, the sample is examined (for example, visually scanned) to identify a region that has a concentration of tumour cells that has amplification of the gene. Gene copy number in the cells with highest amplification in the selected region is then counted. In other examples, such as when the sample is a tumour core (such as a tumour microarray), most of the sample is visible in the field of view under low power magnification and the individual cells (such as tumour cells) with the strongest signal (for example, highest amplification signal under high power) are separately identified for counting. In particular examples, the cells chosen for counting the gene copy number may be non-consecutive cells, such as cells that are not adjacent to or in contact with one another. In other examples, at least some of the cells chosen for counting the gene copy number may be consecutive cells, such as cells that are adjacent to or in contact with one another.

The disclosed methods include counting the number of ISH signals (such as fluorescent, coloured, or silver spots) for the gene in the identified cells. The methods may also include counting the number of ISH signals (such as fluorescent, coloured or silver spots) for a reference (such as a chromosome-specific probe) in the identified cells. In some examples, the number of spots per cells is distinguishable in the identified cells and the number of spots are counted (or enumerated) and recorded. In other examples, one or more of the identified cells may include a cluster, which is the presence of multiple overlapping signals in a nucleus that cannot be counted (or enumerated). In particular examples, the number of copies of the gene (or chromosome) may be estimated by the person (or computer, in the case of an automated method) scoring the slide. For example, one of skill in the art of pathology may estimate that a cluster contains a particular number of copies of a gene (such as 10, 20, or more copies) based on experience in enumerating gene copy number in a sample. In other examples, the presence of a cluster may be noted as a cluster, without estimating the number of copies present in the cluster.

The number of cells identified for counting is a sufficient number of cells that provides for detecting a change (such as an increase or decrease) in gene copy number. In some examples, the number of cells identified for counting is at least about 20, for example, at least 25, 30, 40, 50, 75, 100, 200, 500, 1000 cells, or more. In a particular example, about 50 cells are counted. In other examples, every cell in the sample or every cell in a microscope field of vision, or in a number of microscope fields (such as at least 2 microscope fields, at least 3, at least 4, at least 5, at least 6 microscope fields, and the like) which contains 3 or more copies of the gene of interest (such as 3, 4, 5, 6, 7, 8, 9, 10, 15, 20, or more) is counted. In some examples, the biological sample is a tumour sample, such as a tumour which potentially includes a gene amplification. Exemplary biological samples include neoplastic cells or tissues, which may be isolated from solid tumours, including lung cancer (e.g., non-small cell lung cancer, such as lung squamous cell carcinoma), breast carcinomas (e.g. lobular and duct carcinomas), adrenocortical cancer, ameloblastoma, ampullary cancer, bladder cancer, bone cancer, cervical cancer, cholangioma, colorectal cancer, endometrial cancer, esophageal cancer, gastric cancer, glioma, granular call tumour, head and neck cancer, hepatocellular cancer, hydatiform mole, lymphoma, melanoma, mesothelioma, myeloma, neuroblastoma, oral cancer, osteochondroma, osteosarcoma, ovarian cancer, pancreatic cancer, pilomatricoma, prostate cancer, renal cell cancer, salivary gland tumour, soft tissue tumours, Spitz nevus, squamous cell cancer, teratoid cancer, and thyroid cancer.

In a preferred embodiment, the determination of the copy number of the CHKA gene is carried out by FISH. The term “fluorescent in situ hybridisation” or FISH has already been defined for the first method of the invention and thus, will not be explained herein in further detail. In a particular embodiment, the FISH is carried out using at least two BACs selected from the group of the CTD-2162L23, RP11-314K20, CTD-2655K5 and CTC-783C1 BACs. The BACs have already been explained in the composition of the invention.

Once the level of amplification of the CHKA gene has been determined, the second method of the invention comprises determining whether the patient will have a favourable or an unfavourable outcome depending on the level of amplification of the CHKA gene. Thus, patients having amplification of the CHKA gene will have an unfavourable outcome whether patients having a normal copy number of the CHKA gene will most likely show a favourable outcome.

The term “unfavourable outcome of the subject” as used in the present invention, refers to a subject whose prognosis is not good and who is likely to develop aggressive, recurrent disease, to develop one or more metastases, to survive for a small amount of time, e.g. 1, 2, 3, 4, or 5 years, to survive a small amount of time without disease progression (e.g., an unfavourable outcome of a subject will be survival of only 1, 2, 3, 4, or 5 years without progression) or to respond negatively to a particular therapy (e.g., chemotherapy).

A good prognosis entails, e.g., survival of a patient for more than 1 year after initial diagnosis (such as more than 2 years or more than 5 years), or survival of a patient for more than 6 months longer (e.g., more than 1 year longer, more than 2 years longer, more than 5 years longer) than the average survival for similarly situated patients. A poor prognosis entails, e.g., survival of a patient for less than 5 years after initial diagnosis (such as less than 2 years or less than 1 year), or survival of a patient less than the average survival for similarly situated patients (such as, about 3 months less than average survival, about 6 months less than average survival, or about 1 year less than average survival). In other examples, a good prognosis further predicts that a neoplasm may be less aggressive (e.g., less rapidly growing, and/or less likely to metastasize). A good prognosis may entail progression-free survival (such as lack of recurrence of the primary tumour or lack of metastasis) of a patient for more than 1 year after initial diagnosis (such as more than 2 years or more than 5 years), or progression-free survival of a patient for more than 6 months longer (e.g., more than 1 year longer, more than 2 years longer, more than 5 years longer) than the average survival for similarly situated patients. A poor prognosis may predict that a neoplasm may be more aggressive (e.g., more rapidly growing and/or more likely to metastasize). A poor prognosis may entail, e.g., progression-free survival of a patient for less than 5 years after initial diagnosis (such as less than 2 years or less than 1 year), or progression-free survival of a patient less than the average survival for similarly situated patients (such as, about 3 months less than average survival, about 6 months less than average survival, or about 1 year less than average survival).

For example, a good prognosis includes a greater than 40 percent chance that the subject will survive to a specified time point (such as one, two, three, four or five years), and/or a greater than 40 percent chance that the tumour will not metastasize. In several examples, a good prognosis indicates that there is a greater than 50 percent, 60 percent, 70 percent, 80 percent, or 90 percent chance that the subject will survive and/or a greater than 50 percent, 60 percent, 70 percent, 80 percent or 90 percent chance that the tumour will not metastasize. Similarly, a poor prognosis includes a greater than 50 percent chance that the subject will not survive to a specified time point (such as one, two, three, four or five years), and/or a greater than 50 percent chance that the tumour will metastasize. In several examples, a poor prognosis indicates that there is a greater than 60 percent, 70 percent, 80 percent, or 90 percent chance that the subject will not survive and/or a greater than 60 percent, 70 percent, 80 percent or 90 percent chance that the tumour will metastasize

In a preferred embodiment, the parameters to be measured are disease-free survival and overall survival.

In some examples, an increased CHKA gene copy number includes CHKA gene copy number per nucleus (such as average CHKA gene copy per nucleus) in the sample of greater than about two copies of the gene per nucleus (such as greater than 2, 3, 4, 5, 10, or 20 copies). In other examples, an increased CHKA gene copy number includes a ratio of CHKA gene copy number to Chromosome 11 copy number (such as an average CHKA gene: Chromosome 11 ratio) in the sample of greater than about 2 (such as a ratio of greater than 2, 3, 4, 5, 10, or 20). In further examples, an increased CHKA copy number includes an increase in CHKA gene copy number relative to a control (such as an increase of about 1.5-fold, about 2-fold, about 3-fold, about 5-fold, about 10-fold, about 20-fold, or more).

In other examples, both the CHKA gene and Chromosome 11 DNA (such as

Chromosome 11 centromeric DNA) are detected in a sample from the subject, for example by ISH. Chromosome 11-specific probes are well known in the art and include commercially available probes, such as Zymed SPOT-Light® Chromosome 11 Centromeric Probe (Invitrogen) or the Chromosome 11 Control Probe (Empire Genomics). The CHKA gene and Chromosome 11 DNA may be detected on the same sample (for example, on a single slide or tissue section, such as a dual colour assay) or in different samples from the same subject (for example, CHKA gene is detected on one slide and Chromosome 11 DNA is detected on a matched slide from the same subject, such as a single colour assay). The CHKA gene and Chromosome 11 DNA are detected with two different detectable labels for dual colour assay (such as two different fluorophores, two different chromogens, or a chromogen and metal nanoparticles). The CHKA gene and Chromosome 11 DNA may be detected with the same label for single colour assay. The CHKA gene and Chromosome 11 DNA copy number may be determined by counting the number of fluorescent, coloured, or silver spots on the chromosome or nucleus. A ratio of CHKA gene copy number and Chromosome 11 DNA number is then determined.

Thus, in a particular embodiment, if the sample of the subject which has been amplified has more than 2 copies of the CHKA gene, it is indicative of an unfavourable outcome of the subject. In a particular embodiment, if the sample of the subject which has been amplified has more than 4 copies of the CHKA gene, it is indicative of an unfavourable outcome of the subject.

The second method of the invention may further comprise the determination of the copy numbers of other genes which are known to be amplified in cancer cells. Examples include, but are not limited to IGF1R (15q26.3; e.g., GENBANK™ Accession No. NC_{—}000015, nucleotides 97010284-97325282), EGFR (7p12; e.g., GENBANK™ Accession No. NC_{—}000007, nucleotides 55054219-55242525), HER2 (17q21.1; e.g., GENBANK™ Accession No. NC_{—}000017, nucleotides 35097919-35138441), C-MYC (8q24.21; e.g., GENBANK™ Accession No. NC_{—}000008, nucleotides 128817498-128822856), TOP2A (17q21-q22; e.g., GENBANK™ Accession No. NC_{—}000017, complement, nucleotides 35798321-35827695), MET (7q31; e.g., GENBANK™ Accession No. NC 000007, nucleotides 116099695-116225676), FGFR1 (8p1 1.2-p1 1.1; e.g., GENBANK™ Accession No. NC_{—}000008, complement, nucleotides 38387813-38445509), FGFR2 (10q26; e.g., GENBANK™ Accession No. NC_{—}000010, complement, nucleotides 123227845-123347962), MDM2 (12q14.3-q15; e.g., GENBANK™ Accession No. NC_{—}000012, nucleotides 67488247-67520481), KRAS (12p12.1; e.g. GENBANK™ Accession No. NC_{—}000012, complement, nucleotides 25249447-25295121), and TYMS (18p1 1.32; e.g., GENBANK™ Accession No. NC OOOO 18, nucleotides 647651-663492), wherein the amplification in the CHKA gene and in one or more of said genes is indicative of a worse prognosis.

The following example is provided as merely illustrative and is not to be construed as limiting the scope of the invention.

### EXAMPLE

### I. Materials and Methods

1. Development of Cytogenetic Probes to Analyze Gene Amplification or Translocation

The probes were generated using BACs (Bacterial Artificial Chromosomes) which contained the CHKA gene. All information about these BACs is available in different public databases such as UCSC (www.genome.ucsc.edu) and Ensembl (www.ensembl.org). A total of 9 BACs were potentially useful for gene amplification, i.e. they contained the whole CHKA gene, and 6 BACs (3 pairs) were useful for translocation analysis, i.e. each pair flanked the CHKA gene. If there is a translocation, the signals of the pair of BACs will be separated in different chromosomes and if there is not translocation, the signals will be joined. Comparing the information about these BACs in the different public databases, 8 BACs were discarded because their location were mistaken (they were not in chromosome 11q13 where CHKA is located) or they hybridized to chromosomes other than 11q13. After this initial filtering step, 7 BACs were finally selected (5 for gene amplification and 2 for translocation analysis). The BACs selected were commercially available and were the following (the size and the sequence of the fragment is also indicated):

BACs for gene amplification
RP11-314K20 (193 kb) (SEQ ID NO: 2)
RP11-110E14 (171 kb) (SEQ ID NO: 5)
CTC-783C1 (154 kb) (SEQ ID NO: 4)
CTD-2162L23 (126 kb) (SEQ ID NO: 1)
CTD-2655K5 (191 kb) (SEQ ID NO: 3)
Translocation BACs
CTD-2061I7 (136 kb) (SEQ ID NO: 6)
CTD-3054O2 (94 kb) (SEQ ID NO: 7)

The BACs were grown and the DNA extracted. The fluorescence labelling was performed using the nick translation reaction.

2. DNA Labelling by Nick Translation

After labelling the seven BACs preselected in the previous step using nick-translation, the BACs were evaluated by FISH in normal cells. The results confirmed that they hybridized only to chromosome 11q13.

3. Fluorescence In Situ Hybridisation (FISH)

The technique allows the detection and localization of specific sequences of DNA or RNA using chromosomal preparations, cell extensions and preparations of tissue embedded in paraffin. In the last years its use has increased considerably as a complement of the conventional cytogenetics techniques. This method is based on using three types of probes: centromeric probes, chromosomal painting probes and single sequence probes (also known as specific locus probes). The probes used in the present example belong to this last type of probes which hybridize with the DNA of a specific genomic region corresponding to a gene or chromosomal region involved in different alterations (gene amplification, translocation between two genes or inversion that affects to one or several genes).

4. Analysis of Normal and Tumour Cell Lines to Optimize the Conditions of Hybridisation for the Probes Containing CHKA

Metaphasic chromosomes from normal cell lines and metaphasic chromosomes from tumour cell lines with different expression levels of CHKA were used in the assays.

The tumour cell lines are listed herein:

| Bladder | Breast | Lung | Colon |
|---|---|---|---|
| UROTSA | HMEC (primary) | H460 | DLD1 |
| (immortalized) | | | |
| TCC-Sup | MCF7 | H510 | HT29 |
| SW-780 | MDA-MB-231 | H82 | |
| J-82 | SK-BR3 | | |

5. Cytogenetics Analysis of Paraffin-Embedded Normal and Tumour Tissues

In hospitals, the main type of material used to diagnose and predict the outcome of the patient is paraffin-embedded tissue. That is the reason why a cytogenetics analysis was carried out in normal and tumour tissues embedded in paraffin from patients with NSCLC.

Optimization of FISH to analyze paraffin-embedded tissues was more difficult since the probes signals were very weak when they were used individually. Therefore, it was necessary to combine four out of the five probes designed to detect amplification to improve the hybridisation signal. Selected BACs were 2162123, 314k20, 2655k5 and 783c1, labelled all of them in red. Following this strategy, the hybridisation signals obtained were good enough to perform a retrospective study in lung cancer. A specific probe for the centromere of chromosome 11, labelled in green, was used to evaluate the number of this chromosome at same time that the copy number of CHKA was analyzed. This approach helped to discern between aneuploidy (more than two chromosomes 11) and gene amplification (copies not necessarily located in chromosome 11).

Tumours from a total of 50 patients with non small cells lung cancer (NSCLC) were collected and they were included in 3 different tissue microarrays, where control samples were also included. Control samples were normal lung tissue and normal liver tissue because the CHKA basal level is very high in this tissue. All the samples were analyzed as duplicate.

### II. Results

1. Optimization of the Conditions of Hybridisation for the Probes Containing CHKA

First of all, it was shown that the probes generated hybridized to chromosome 11q13 where CHKA gene is located. After that, the hybridisation conditions were optimized to obtain the best results. To do that, different hybridisations using the seven probes over metaphasic chromosomes of different healthy individuals were performed. Once it was confirmed that the probes worked correctly, different tumour cell lines were analyzed as a first approach to dilucidate whether gene amplification or translocation could be mechanisms of CHKA over-expression.

When it was possible, the results obtained by FISH in tumour cell lines were compared with normal cell lines from the same tissue. FISH analysis showed a mean of 3-4 extra signals in tumour cell lines comparing to the 2 normal signals in primary cells (Table 1). These extra signals correlated to high level of CHKA protein, in fact the lung tumour cell line called H460 and the colon tumour cell line known as DLD1, both of them with low protein expression, presented a normal hybridisation pattern. It means they had only 2 gene copies. In general the correlation between gene copy number and protein expression of CHKA were good. At the same time, using the probes to detect gene translocation, this mechanism was excluded as explanation of CHKA over-expression.

TABLE 1
Results obtained by FISH using the probes to analyze
gene amplification or gene translocation.

| | | | |
|---|---|---|---|
| | CHKA | | |
| Cell lines | levels | Gene amplification | Translocation |

Bladder

| | | | |
|---|---|---|---|
| UROTSA | | 2 signals in chr 11q13 | No |
| (primary) | | | |
| TCC-Sup | Chok ↑ | 4 signals in chr 11q13 | No |
| SW-780 | Chok ↑↑ | 4 signals in chr 11q13 | No |
| | | (1chr was 11p-) | |
| J-82 | Chok ↑↑ | 4 signals in chr 11q13 | No |

Breast

| | | | |
|---|---|---|---|
| HMEC (primary) | | 2 signals in chr 11q13 | No |
| MCF7 | Chok ↑ | 4 signals | No |
| | | (2 chr 11, 1 chr19 & | |
| | | 1 marker) | |
| MDA-MB-231 | Chok ↑↑ | 3 signals in chr 11q13 | No |
| SK-BR3 | Chok ↑↑ | 4 signals in chr 11q13 | No |

Lung

| | | | |
|---|---|---|---|
| H460 | Chok ↑ | 2 signals in chr 11q13 | No |
| H510 | Chok ↑↑ | 3 signals in chr 11q13 | No |
| H82 | Chok ↑↑ | 2 signals in chr 11q13 | No |

Colon

| | | | |
|---|---|---|---|
| DLD1 | Chok ↑ | 2 signals in chr 11q13 | No |
| HT29 | Chok ↑↑ | 4 signals in chr 11q13 | No |

2. Cytogenetics Analysis of Paraffin-Embedded Normal and Tumour Tissues

Finally, 48 lung tumour samples were analyzed by FISH using the assayed probes and the hybridisation conditions optimized. The conclusion was that 69% of them showed more than 2 copies of chromosome 11q13 (33 over 48). Near 90% of these (29 over 33) had between 3 and 6 gene copies and all of them presented more than 2 centromeric signals, it means more than 2 chromosomes 11.

3. Survival Analysis of Patients NSCLC With Different Gene Copy Number by FISH

3.1. Statistical Analyses

The Kaplan-Meier method was used to estimate overall and relapse-free survival. Only death from recurrence of lung cancer was considered in the study. The effect of the different factors on tumour-related recurrence and survival was assessed by the log-rank test for univariate analysis. To assess the effect of CHKA expression on survival, with adjustment for potential confounding factors, proportional hazard Cox regression modelling was used. Hazard ratios (HR) and 95% confidence intervals (95% CI) were calculated from the Cox regression model. All reported p values were two-sided. Statistical significance was defined as p<0.05. Statistical analyses were done using the SPSS software (version 17.0). In Table 2 it is disclosed the statistics of the patients included in the study.

TABLE 2
Characteristics of patients included in the study

| | n (%) | |
|---|---|---|
| Age | 43-82 years | |
| | (median 63.5) | |
| Sex | | |
| Men | 42 | (87.5%) |
| Women | 6 | (12.5%) |
| Histology | | |
| Squamous cell carcinoma | 32 | (66.7%) |
| Adenocarcinoma | 10 | (20.8%) |
| Other | 6 | (12.5%) |
| Stage | | |
| I_{A} | 3 | (6.3%) |
| I_{B} | 19 | (39.6%) |
| II_{A} | 2 | (4.2%) |
| II_{B} | 5 | (10.4%) |
| III_{A} | 8 | (16.7%) |
| III_{B}-IV | 7 | (14.6%) |
| Total | 48 | |
| Relapse | | |
| No | 31 | (64.6%) |
| Yes | 14 | (29.2%) |
| Unknown | 3 | (6.3%) |

3.2. Prognostic Value of CHKA Copy Number in NSCLC

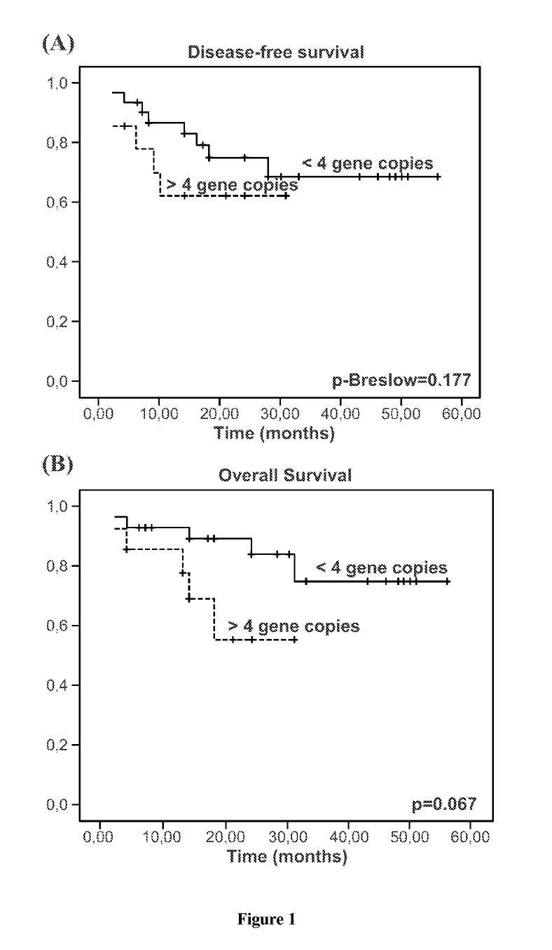

To study whether CHKA copy number was associated with clinical outcome of patients with NSCLC, 48 surgical specimens of NSCLC were analyzed by FISH. It was not found relation between CHKA copy number and the available clinical-pathologic parameters of the patients (stage, histological degree, age or sex). To analyse whether increased copy number of CHKA observed in a group of the tumours was associated with the clinical outcome of the patients, the patients were divided into two groups (the first one with tumour samples comprising less or equal to 4 gene copies and the second one with more than 4 gene copies). Under these conditions, 14 out of the 48 (29%) tumour samples analysed for CHKA copy number corresponded to the second group. Patients with increased gene copy number showed worse survival from lung cancer and relapse-free survival than those with lower gene copies (FIG. 1).

An association between less than 4 CHKA copies and improved lung-cancer specific survival was noted (p=0.067). The mean survival of patients with less than 4 gene copies was 46.9 months (39.65-54.1) whereas the mean survival of those with more than 4 gene copies decreased to 22.3 months (16.29-28.36).

Taken together, these results suggest that CHKA copy number is closely associated with relapse-free and overall survival among patients with NSCLC. Thus, the analysis of CHKA copy number by FISH could be a new prognostic factor that could be used to help identify patients with cancer with favourable prognosis who could receive less aggressive treatment options or avoid adjuvant systemic treatment, specifically patients with NSCLC.

1. A composition comprising at least two polynucleotides selected from the group consisting of:
(a) a polynucleotide comprising the human genomic fragment present in the CTD-2162L23 BAC (SEQ ID NO:1) or a variant thereof which hybridizes under stringent conditions to said polynucleotide,
(b) a polynucleotide comprising the human genomic fragment present in the RP11-314K20 BAC (SEQ ID NO:2) or a variant thereof which hybridizes under stringent conditions to said polynucleotide,
(c) a polynucleotide comprising the human genomic fragment present in the CTD-2655K5 BAC (SEQ ID NO:3) or a variant thereof which hybridizes under stringent conditions to said polynucleotide and
(d) a polynucleotide comprising the human genomic fragment present in the CTC-783C1 BAC (SEQ ID NO:4) or a variant thereof which hybridizes under stringent conditions to said polynucleotide
wherein said polynucleotides are not associated with all or a portion of a polynucleotide in which the isolated polynucleotide is found in nature.

2. Composition according to claim 1 wherein one or more of the polynucleotides are provided in BACs.

3. Composition according to claim 2 wherein the BACs are the CTD-2162L23, RP11-314K20, CTD-2655K5 and CTC-783C1 BACs.

4-6. (canceled)

7. An in vitro method for predicting the outcome of a subject suffering from cancer comprising detecting whether the CHKA gene is amplified in a sample from said subject, wherein amplification of the CHKA gene is indicative of an unfavourable outcome of the subject.

8. Method according to claim 7 wherein detecting the amplification of the CHKA gene comprises determining the copy number of the CHKA gene.

9. Method according to claim 8 wherein more than 2 copies of the CHKA gene is indicative of the subject suffering from cancer or of an unfavourable outcome of the subject.

10. Method according to claim 7 wherein the detection of the amplification is carried out using as at least two BACS selected from the group of the CTD-2162L23, RP11-314K20, CTD-2655K5 and CTC-783C1 BACs.

11. Method according to claim 7 wherein the cancer is selected from the group of breast cancer, colon cancer, lung cancer, bladder cancer and prostate cancer.

12. Method according to claim 11 wherein lung cancer is non-small cell lung cancer (NSCLC).

13. Method according to claim 12, wherein the NSCLC is selected from squamous cell carcinoma of the lung, large cell carcinoma of the lung and adenocarcinoma of the lung.

14. Method according to claim 13, wherein the NSCLC is advanced NSCLC, preferably, stage IIIA, IIIB or IV NSCLC.

15. Method according to claim 7, wherein the sample is a tumour tissue sample.

16. A method for determining the copy number of the CHKA gene which comprises:
a. contacting a sample containing genetic material with a composition comprising at least two polynucleotides selected from the group consisting of:
(i) a polynucleotide comprising the human genomic fragment present in the CTD-2162L23 BAC (SEQ ID NO:1) or a variant thereof which hybridizes under stringent conditions to said polynucleotide,
(ii) a polynucleotide comprising the human genomic fragment present in the RP11-314K20 BAC (SEQ ID NO:2) or a variant thereof which hybridizes under stringent conditions to said polynucleotide,
(iii) a polynucleotide comprising the human genomic fragment present in the CTD-2655K5 BAC (SEQ ID NO:3) or a variant thereof which hybridizes under stringent conditions to said polynucleotide and
(iv) a polynucleotide comprising the human genomic fragment present in the CTC-783C1 BAC (SEQ ID NO:4) or a variant thereof which hybridizes under stringent conditions to said polynucleotide
wherein said polynucleotides are not associated with all or a portion of a polynucleotide in which the isolated polynucleotide is found in nature wherein said contacting is carried out under conditions adequate for the hybridisation of the polynucleotides forming part of the composition with the CHKA gene or genes present in the sample; and
b. determining the copy number of the CHKA gene based on the hybridisation of the composition with the sample containing genetic material.

17. Method according to claim 16 wherein the determination of the copy number of the CHKA gene is carried out by in situ hybridisation.

18. Method according to claim 16 wherein one or more of the polynucleotides of the composition used in step a. are provided in BACs.

19. Method according to claim 18 wherein the contacting step is carried out using a composition comprising the polynucleotides comprising the genomic fragment present in the CTD-2162L23, RP11-314K20, CTD-2655K5 and CTC-783C1 BACs.

20. Method according to claim 19 wherein the contacting step is carried out using a composition comprising at least two BACs selected from the group consisting of the CTD-2162L23, RP11-314K20, CTD-2655K5 and CTC-783C1 BACs.
